(19) United States
(12) Patent Application Publication
(10) Pub. No.: US 2014/0025086 A1
(43) Pub. Date: Jan. 23, 2014

(54) DEVICE AND METHOD FOR CAPTURING GUIDEWIRES

(71) Applicants: Dan Rottenberg, Haifa (IL); Ron Sacher, Herzelia (IL)

(72) Inventors: Dan Rottenberg, Haifa (IL); Ron Sacher, Herzelia (IL)

(73) Assignee: The Spectranetics Corporation, Colorado Springs, CO (US)

(21) Appl. No.: 13/390,146

(22) PCT Filed: Dec. 12, 2011
(86) PCT No.: PCT/US2011/064302

§ 371 Date: Feb. 13, 2012

Related U.S. Application Data
(60) Provisional application No. 61/516,906, filed on Apr. 11, 2011.
(60) Provisional application No. 61/571,856, filed on Jul. 7, 2011.
(60) Provisional application No. 61/575,160, filed on Aug. 17, 2011.
(60) Provisional application No. 61/573,935, filed on Sep. 15, 2011.
(60) Provisional application No. 61/626,183, filed on Sep. 22, 2011.

(51) Int. Cl. A61B 17/221; A61B 17/50

(52) U.S. Cl. CPC A61B 17/221; A61B 17/50
USPC 606/127

(57) ABSTRACT

Device and method for capturing guidewires inside blood vessels. The disclosed device enables retrieval of guidewires that need to be pulled from the body at a different location than the guidewire entering location, such as guidewires that have entered in a retrograde direction.

### FIELD OF THE INVENTION

The present invention generally relates to a device and a method for retrograde guidewire retrieval during blood vessel angioplasty, such as below the knee (BTK) blood vessels and other blood vessels (e.g., femoral or iliac), which are partially or totally occluded.

### BACKGROUND OF THE INVENTION

A chronic total occlusion (CTO) is an arterial vessel blockage that prevents blood flow beyond the obstruction. CTO's typically occur in coronary, peripheral, pediatric, and other small arteries. In the coronary and peripheral arteries, they result from the same underlying cause—atherosclerosis.

Endovascular therapy for arteries below the knee has emerged as a promising revascularization technique for patients with critical limb ischemia (CLI). However, when employing standard angioplasty techniques, angioplasty of BTK arteries fails to achieve revascularization in up to 20% of cases. The main cause for failure is the inability to penetrate the plaque's proximal cap with the guidewire.

A new technique of approaching the plaque from below—known as the retrograde approach—is often used to pass the guidewire through the plaque from the other direction. This approach has high success rates, but is technically challenging to perform and has its own complications, especially the danger of vessel perforation.

In order to use the retrograde technique, the clinician must puncture the small target artery with a needle—usually smaller than a 21 gauge needle. The clinician relies on several angiographic images to aim the needle into the artery, and verifies proper needle tip location by observing blood flow exiting from the needle's proximal end.

Puncturing small arteries is not easy; it requires proper manipulation of the C-arm and a gentle needle stick to avoid arterial perforation. Once a small sheath or direct (sheath-less) guidewire is inserted within the needle into the artery, the needle can be removed.

If retrograde passage of the guidewire through the occlusion is successful transluminally, the guidewire must be snared and pulled through the antegrade or cross-over sheath, using “Lasso” snaring devices or a simple 5 or 6 Fr (1.7-2.0 mm) support catheter. Thereafter, relatively large balloons and stents can be delivered without damaging the small retrograde puncture hole, preventing the risk of small blood vessel perforation. For example, the tibial-peroneal vessels are small, having diameters of about 2 mm (6 Fr) to 3 mm (9 Fr). Therefore, inserting a 6 Fr or even a 5 Fr sheath needed for balloons and stent delivery, will perforate the artery.

However, although using a “Lasso” device is feasible it is not easy; it requires gentle manipulation, and the guidewire tip often becomes damaged and cannot be used.

Retrieval of the guidewire with a small support catheter can be very difficult; it takes a long time with many attempts, and has a low rate of success, due to the small catheter lumen compared to the relatively larger vessel lumen at the wire exit area.

Such guidewire capturing is also needed when treating iliac arteries, which are relative large, but guidewires are often inserted through distant arteries, like radial arteries, to avoid iliac dissection. Such guidewires must be captured near the occlusion for easy delivery of balloons and stents.

If retrograde passage of the guidewire through the occlusion is performed via a subintimal approach, the true lumen must be reentered, either spontaneously or with a reentry device. Current reentry devices are big, stiff, and require at least a 6 Fr sheath through which to pass. Therefore, they cannot be used from a retrograde approach.

### SUMMARY OF THE INVENTION

The present invention is directed to a device and a method for retrograde guidewire retrieval during blood vessel angioplasty. The invention seeks to improve the physician's technique in treating blood vessel occlusions, and can be used when crossing both new, soft plaque and old, hard plaque.

In accordance with a non-limiting embodiment of the invention, the retrograde approach may be performed with the following steps:
a. Insert needle tip into the (small) blood vessel.
b. Insert a standard 0.014″, 0.018″ or 0.035″ guidewire through the needle into the artery, and remove the needle and needle holder.
c. Attempt crossing the plaque with the guidewire alone. If not successful, use the flexible sheath introducer as a support catheter for the guidewire. If a sheath-less technique is used, use a support catheter to support the guidewire passage through the plaque.
d. If the guidewire ultimately crosses the plaque successfully, and must be captured from above/other side into the cross-over or antegrade sheath, insert the guidewire retrieval catheter of the present invention from the other side, to funnel the guidewire out through the sheath via the special conical tip of the guidewire retrieval catheter.

### BRIEF DESCRIPTION OF THE DRAWINGS

The present invention will be understood and appreciated more fully from the following detailed description taken in conjunction with the drawings in which:

FIG. 1 is a simplified illustration of a guidewire retrieval catheter, having an elastomeric, conical funnel tip, inserted in an artery, constructed and operative in accordance with a non-limiting embodiment of the present invention.

FIG. 2 is a simplified illustration of the guidewire retrieval catheter of FIG. 1 with a cover sheath, in accordance with a non-limiting embodiment of the present invention.

FIG. 3 is a simplified illustration of a guidewire retrieval catheter, having an elastomeric, conical funnel tip and blood flow blocking balloon, constructed and operative in accordance with a non-limiting embodiment of the present invention.

FIG. 4 is a simplified illustration of a funnel insertion device, constructed and operative in accordance with a non-limiting embodiment of the present invention.

FIG. 5 is a simplified illustration of a guidewire retrieval catheter, having a conical funnel tip made of a shape memory material, constructed and operative in accordance with a non-limiting embodiment of the present invention.

### DETAILED DESCRIPTION OF EMBODIMENTS

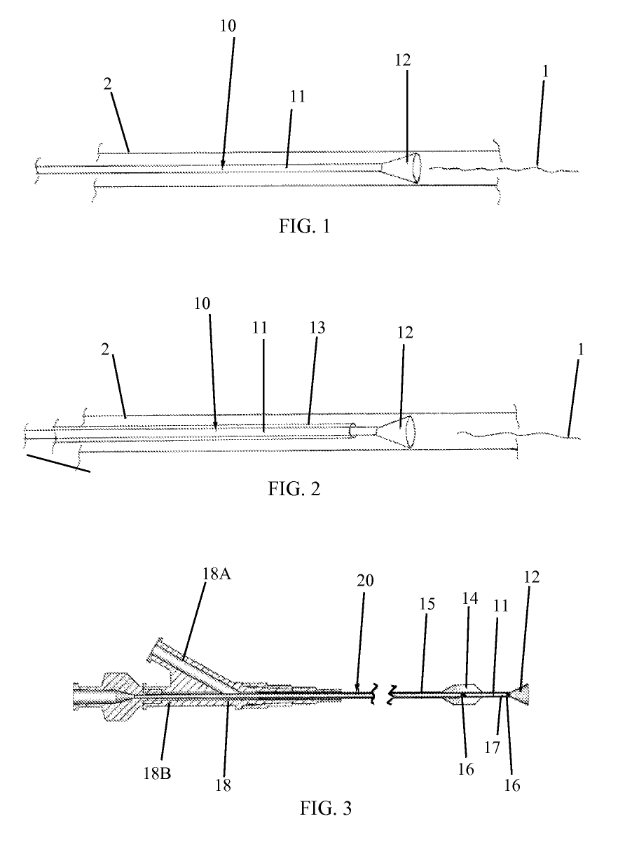

Reference is now made to FIG. 1, which illustrates a guidewire retrieval device 10, constructed and operative in accordance with an embodiment of the present invention. The guidewire retrieval device 10 is also referred to as guidewire retrieval catheter 10, but it is understood that the device is not limited to the configuration of a catheter.

The guidewire retrieval device 10 includes a tube 11, constructed from a suitable, biomedically safe material, such as, but not limited to, PEBAX (trade name for a polyether block amide), nylon, or PTFE (polytetrafluoroethylene). A funnel 12 (preferably cone-shaped, but other shapes are possible) is disposed at a distal end of tube 11. Funnel 12 may be constructed from a flexible, elastomeric material, such as, but not limited to, polyurethane or silicone. FIG. 1 shows guidewire retrieval device 10 inserted in a blood vessel 2 from the antegrade direction. Funnel 12 is open capture a guidewire 1 inserted from the retrograde direction. Opposite directions can also be used if desired by the operator. The size of the open mouth of funnel 12 is preferably equal or slightly larger than the blood vessel diameter.

Guidewire 1 is pushed into funnel 12, which directs guidewire 1 into tube 11. Pushing guidewire 1 further allows capturing its distal tip outside of the patient's body, allowing continuation of the procedure from that side.

Reference is now made to FIG. 2. Optionally, retrieval catheter 10 (or any of the other embodiments of the invention) may include a long sheath 13 to cover and protect funnel 12 during insertion into the blood vessel. Sheath 13 is withdrawn to expose funnel 12, after funnel 12 has reached its proper location inside the blood vessel 2.

Reference is now made to FIG. 3, which illustrates a guidewire retrieval catheter 20, including the elastomeric, conical funnel 12 and an additional blood flow blocking balloon 14, constructed and operative in accordance with a non-limiting embodiment of the present invention. As with the embodiment of FIG. 1, guidewire retrieval catheter 20 includes a tube 11.

Balloon 14 is proximal to funnel 12. An additional coaxial tube 15 is added over tube 11 to create a balloon inflation lumen. Double-lumen tube can be used as well, as is known in the art. Balloon 14 is made from a polymer material, such as a compliant or semi-compliant material, e.g., polyurethane or silicone, but can also be made from nylon or stiffer non-compliant materials. The diameter of balloon 14 is preferably similar or slightly larger than that of the blood vessel lumen in its nominal inflation pressure. Balloon 14 blocks blood flow, preventing possible collapse of funnel 12 due to blood flow and pressure that could close or shrink funnel 12.

Balloon 14 is preferably located quite near funnel 12, so as not only to block blood flow, but also to support centering the relative soft funnel 12 in the blood vessel. (Funnel 12 is relative soft to prevent possible damage to blood vessel 2 during insertion of catheter 20.) Since blood flow might collapse the soft funnel, balloon 14 is needed to prevent such funnel collapse. In addition, since blood vessels are not always straight, centering funnel 12 in the blood vessel using inflated balloon 14 is important for proper funnel function, assuring that funnel 12 touches the entire inner circumference of the blood vessel. Such funnel centering is required even if the device is used against the blood flow direction, when blood flow might naturally open the funnel.

Balloon 14 can alternatively be a long, non-compliant balloon that can also be used for blood vessel angioplasty (PTA).

The guidewire retrieval catheter 20, or any of the other embodiments of the invention, may include one or more radio-opaque markers 16, which help the user to verify that guidewire 1 is properly inserted into tube 11. The markers 16 may be positioned on tube 11 near a neck 17 of funnel 12, and/or at the middle of balloon 14 if a short balloon is used, or at both sides of balloon 14 if a long balloon is used.

The proximal side of the retrieval catheter 20 may include a Y connector 18 with female luer connectors, as is known in the art, including a side-arm 18a for balloon inflation, and a central arm 18b for guidewire passage.

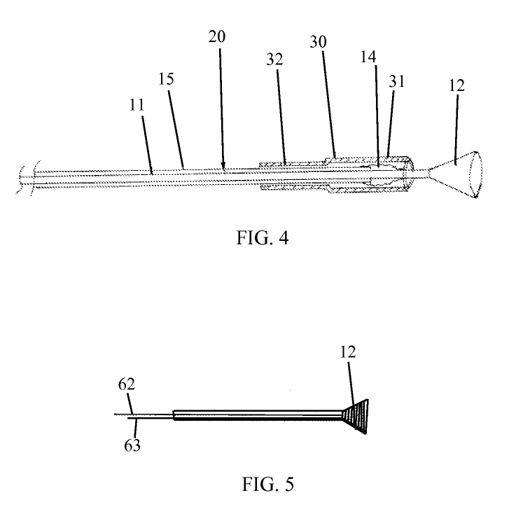

Since funnel 12 is normally open, a device and method of inserting it into the blood vessel through a standard sheath is provided in accordance with an embodiment of the invention as illustrated in FIG. 4.

In one embodiment, a funnel inserting device 30, made of a material, such as, but not limited to, PEBAX, nylon, or PTFE, includes a tube with a distal portion 31 extending from a proximal portion 32. The distal portion 31 has a larger diameter than the proximal portion 32. Proximal portion 32, which may be made from the same tube as distal portion 31, but heat shrunk to a smaller diameter, has a diameter smaller than balloon 14 when folded. This ensures that the funnel inserting device 30 cannot be pushed distally and out by mistake before inserting funnel 12 into a standard sheath.

Distal portion 31 is used as a balloon protector for balloon 14, and for housing the folded funnel 12 before inserting into standard sheath.

The length of distal portion 31 is preferably the distance between the proximal end of balloon 14 and the distal end of funnel 12, or slightly longer.

Funnel 12 does not necessarily have to be made from an elastomeric material. Reference is now made FIG. 5, in which funnel 12 is made from a shape memory material, such as, but not limited to, nitinol. In the illustrated embodiment, the funnel is made from nitinol wire 62. Wire 62 is connected to a stiffer (thicker) nitinol wire 63 that is used to open or close funnel 12. The distal part of wire 62 is pre-shaped like a cone made from a crowded (zero pitch) spiral wire, which circles in an ever reducing diameter at every circle to create the desired cone shape. To prevent possible damage to the blood vessel, the distal end of wire 62 is rounded, and may circle backwards and connect to the distal end of stiff wire 63.

1. A guidewire retrieval device comprising:
a tube;
a flexible funnel disposed at a distal end of said tube, said funnel being conical with an open mouth facing the distal end of said tube; and
a balloon positioned proximal to said funnel.

2. The guidewire retrieval device according to claim 1, wherein said balloon comprises a blood flow blocking balloon.

3. The guidewire retrieval device according to claim 1, wherein said balloon is located sufficiently near said funnel so as to support centering said funnel in a blood vessel.

4. The guidewire retrieval device according to claim 1, further comprising a withdrawable sheath of sufficient length to cover said funnel during insertion into a blood vessel.

5. The guidewire retrieval device according to claim 1, further comprising a coaxial tube disposed over said tube to create a balloon inflation lumen.

6. The guidewire retrieval device according to claim 1, further comprising at least one radio-opaque marker.

7. The guidewire retrieval device according to claim 6, wherein said at least one radio-opaque marker is positioned on said tube of said funnel.

8. The guidewire retrieval device according to claim 6, wherein said at least one radio-opaque marker is positioned on said tube near a middle portion of said balloon.

9. The guidewire retrieval device according to claim 1, further comprising a funnel inserting device, which comprises a tube with a distal portion extending from a proximal portion, said distal portion being arranged for housing therein said balloon and said funnel before inserting into a sheath.

10. The guidewire retrieval device according to claim 1, wherein said flexible funnel is made of an elastomeric material.

11. The guidewire retrieval device according to claim 1, wherein said flexible funnel is made of a shape memory material.

12. A method for guidewire retrieval comprising:
inserting a guidewire retrieval device in a blood vessel from a first direction, said guide wire retrieval device comprising tubes, a balloon and a flexible, elastomeric funnel disposed at a distal end of one of said tubes, said funnel being conical with an open mouth facing the distal end of said one of said tubes;
inflating said balloon to block blood flow and centering said funnel in a lumen of said blood vessel;
manipulating a guidewire in the blood vessel from a second direction opposite to the first direction, until said guide wire moves into said funnel; and
continuing to move said guide wire through said funnel and said tube until capturing a distal end of said guide wire.

13. The method according to claim 12, wherein said first direction is an antegrade direction and said second direction is a retrograde direction.

14. A guide wire retrieval device comprising:
a tube;
a flexible funnel disposed at a distal end of the tube, the flexible funnel being conical with an open mouth configuration facing distally of the tube;
a balloon located proximal of the funnel;
a balloon inflation lumen; and
a sheath initially covering at least a portion of each of the tube, the flexible funnel and the balloon.

15. The guide wire retrieval device according to claim 14, wherein the balloon is near the flexible funnel.

16. The guide wire retrieval device according to claim 15, wherein the balloon is located sufficiently near the funnel so as to support centering said funnel in a blood vessel.

17. The guide wire retrieval device according to claim 14, further comprising at least one radio-opaque marker.

18. The guide wire retrieval device according to claim 17, wherein the at least one radio-opaque marker is positioned on the tube near a neck of the funnel.

19. The guide wire retrieval device according to claim 17, wherein the at least one radio-opaque marker is positioned on the tube near a middle portion of the balloon.

20. The guide wire retrieval device according to claim 14, further comprising a funnel inserting device, which comprises a tube with a distal portion extending from a proximal portion, the distal portion being arranged for housing therein the balloon and the funnel before inserting into a sheath.
